SEQUENCE LISTING (8 sequences)

SEQ ID NO: 1 - DNA, 2856 bp, homo sapiens
atgccgggcccgctagggctgctctgcttcctcgccctggggctgctcggctcggccgggcccagcggcgcggcgccgcctctctgcgcggcgccctgcagctgcgacggcgaccgtcgggtggactgctccggaaaggggttgacggccgtaccggagggtctcagcgccttcacccaagcactggatatcagtatgaacaatatcacccagttaccagaagatgcatttaagagtttcccatttctagaggagctacaactggctggtaacgacctttctcttatccatccaaaagccttgtctgggctgaaagaactcaaagtcctaacactccagaataatcagttgagaacagtgcccagtgaagccattcacggactgagtgctttgcagtctttacgcttagatgccaaccatattacctcagtcccggaggacagttttgaagggcttgtccagttacgccatctgtggctggatgacaacagcttgacggaagtgcccgtgcgtcccctcagcaacctgccaaccctgcaggcgctgaccttggctctcaacaacatctcaagcatccctgacttcgctttcaccaacctttcaagcttggtggttctgcatctgcataacaataaaattaaaagcctcagtcaacactgttttgatggactagataacctggaaaccttggacttgaattacaattacttggatgagtttcctcaggctattaaagcccttcccagccttaaagagctgggatttcacagtaattctatttctgttattcctgatggagcatttggtggtaatccactgctaagaactattcatttgtatgataatcctctgtcttttgtggggaactcagcatttcacaacctgtctgatctgcattgcttagtcattcgtggtgcaagcctggtgcagtggttccccaatctgaccggaactgtccatttggagagtctaaccttgacagggacaaaaataagcagcatacctgatgatctgtgccaaaaccaaaagatgctgaggactctggacttatcttataacaatataagagaccttccaagttttaatggttgtcgtgcattggaagaaatttcattgcagcgtaatcaaatctccctaataaaggaaaatacttttcaaggcctaacatctctaaggattctagatctgagtagaaacctgatccgtgaaattcacagtggagcttttgcgaagcttgggacaattactaacctggatgtaagtttcaatgaattaacttcatttcctacggaaggcctaaatgggctcaatcaactaaagcttgtgggtaacttcaagctgaaagacgccttggcagccagagactttgctaatctcaggtctctatcagtaccatatgcttatcagtgttgtgcattttgggggtgtgactctttatgcaaattaaacacagaagataacagcccccaagaacacagtgtgacaaaagagaaaggtgctacagatgcagcaaatgtcaccagcactgctgagaacgaagaacatagccaaataattatccactgtacaccttcaacaggtgctttcaagccctgtgaatatttactgggaagctggatgattcgccttacagtgtggttcattttcctggtcgccttgcttttcaacctgcttgtcattttaacagtgtttgcgtcttgttcatcactgcctgcctccaaactcttcataggcttgatttctgtgtctaacttactcatgggcatctatactggcatccttacttttcttgatgctgtgtcctggggccgatttgccgaatttggcatttggtgggaaactggcagcggctgcaaggtagccgggtctctggcagtcttctcctcagagagcgctgtattcctattaacactggcagctgtggaaagaagcgtatttgcaaaggatttgatgaaacacgggaagagcagtcacctcagacagttccaggtggccgccctcttagctttgctgggtgccgcagtggcaggctgcttcccccttttccacggagggcaatattctgcatcgcccttgtgtttgccgtttcctacaggagaaaccccatcgttaggattcactgtgaccttagtgctattaaactcactggcatttttactaatggccattatctacactaaactatactgcaacttagagaaggaggacctgtcggaaaactcccagtctagcgtgattaagcacgttgcctggctcatcttcacaaactgcatcttcttctgccctgttgcatttttctcatttgcaccattgatcacggcaatctccatcagccccgagataatgaagtctgttacactgatattcttcccgttgcctgcttgcctgaatccggtcctgtatgttttcttcaacccaaagtttaaagaagactggaagctactgaagcggcgtgttaccaggaaacacggatctgtttcagtttccatcagcagccaaggcggttgtggggaacaggatttctactatgactgtggcatgtattcccacttgcagggtaacctgactgtctgtgactgctgtgagtcatttcttttgacaaaaccagtatcatgcaaacacttaataaaatcgcacagttgtcctgtattgacagcggcctcttgccagaggccagaggcctactggtctgattgtggtacacagtcagcccattctgactatgcagatgaagaagattcctttgtctcagacagctctgaccaggtgcaggcctgtggacgagcctgcttctaccagagtcgtggattccctctggtgcgctatgcttacaatctacagagagtcagagactga

SEQ ID NO: 2 - protein, 951 aa, human
MPGPLGLLCFLALGLLGSAGPSGAAPPLCAAPCSCDGDRRVDCSGKGLTAVPEGLSAFTQALDISMNNITQLPEDAFKSFPFLEELQLAGNDLSLIHPKALSGLKELKVLTLQNNQLRTVPSEAIHGLSALQSLRLDANHITSVPEDSFEGLVQLRHLWLDDNSLTEVPVRPLSNLPTLQALTLALNNISSIPDFAFTNLSSLVVLHLHNNKIKSLSQHCFDGLDNLETLDLNYNYLDEFPQAIKALPSLKELGFHSNSISVIPDGAFGGNPLLRTIHLYDNPLSFVGNSAFHNLSDLHCLVIRGASLVQWFPNLTGTVHLESLTLTGTKISSIPDDLCQNQKMLRTLDLSYNNIRDLPSFNGCRALEEISLQRNQISLIKENTFQGLTSLRILDLSRNLIREIHSGAFAKLGTITNLDVSFNELTSFPTEGLNGLNQLKLVGNFKLKDALAARDFANLRSLSVPYAYQCCAFWGCDSLCKLNTEDNSPQEHSVTKEKGATDAANVTSTAENEEHSQIIIHCTPSTGAFKPCEYLLGSWMIRLTVWFIFLVALLFNLLVILTVFASCSSLPASKLFIGLISVSNLLMGIYTGILTFLDAVSWGRFAEFGIWWETGSGCKVAGSLAVFSSESAVFLLTLAAVERSVFAKDLMKHGKSSHLRQFQVAALLALLGAAVAGCFPLFHGGQYSASPLCLPFPTGETPSLGFTVTLVLLNSLAFLLMAIIYTKLYCNLEKEDLSENSQSSVIKHVAWLIFTNCIFFCPVAFFSFAPLITAISISPEIMKSVTLIFFPLPACLNPVLYVFFNPKFKEDWKLLKRRVTRKHGSVSVSISSQGGCGEQDFYYDCGMYSHLQGNLTVCDCCESFLLTKPVSCKHLIKSHSCPVLTAASCQRPEAYWSDCGTQSAHSDYADEEDSFVSDSSDQVQACGRACFYQSRGFPLVRYAYNLQRVRD

SEQ ID NO: 3 - DNA, 2082 bp, homo sapiens
  misc_feature 768: n = A,T,C or G
ctacatctccataacaatagaatccactccctgggaaagaaatgctttgatgggctccacagcctagagactttagatttaaattacaataaccttgatgaattccccactgcaattaggacactctccaacttaaaggaactaggatttcatagcaacaatatcaggtcgatacctgagaaagcatttgtaggcaacccttctcttattacaatacatttctatgacaatcccatccaatttgttgggagatctgcttttcaacatttacctgaactaagaacactgactctgaatggtgcctcacaaataactgaatttcctgatttaactggaactgcaaacctggagagtctgactttaactggagcacagatctcatctcttcctcaaaccgtctgcaatcagttacctaatctccaagtgctagatctgtcttacaacctattagaagatttacccagtttttcagtctgccaaaagcttcagaaaattgacctaagacataatgaaatctacgaaattaaagttgacactttccagcagttgcttagcctccgatcgctgaatttggcttggaacaaaattgctattattcaccccaatgcattttccactttgccatccctaataaagctggacctatcgtccaacctcctgtcgtcttttcctataactgggttacatggtttaactcacttaaaattaacaggaaatcatgccttacagagctggatatcatctgaaaactttccagaactcaaggtnatagaaatgccttatgcttaccagtgctgtgcatttggagtgtgtgagaatgcctataagatttctaatcaatggaataaaggtgacaacagcagtatggacgaccttcataagaaagatgctggaatgtttcaggctcaagatgaacgtgaccttgaagatttcctgcttgactttgaggaagacctgaaagcccttcattcagtgcagtgttcaccttccccaggccccttcaaaccctgtgaacacctgcttgatggctggctgatcagaattggagtgtggaccatagcagttctggcacttacttgtaatgctttggtgacttcaacagttttcagatcccctctgtacatttcccccattaaactgttaattggggtcatcgcagcagtgaacatgctcacgggagtctccagtgccgtgctggctggtgtggatgcgttcacttttggcagctttgcacgacatggtgcctggtgggagaatggggttggttgccatgtcattggttttttgtccatttttgcttcagaatcatctgttttcctgcttactctggcagccctggagcgtgggttctctgtgaaatattctgcaaaatttgaaacgaaagctccattttctagcctgaaagtaatcattttgctctgtgccctgctggccttgaccatggccgcagttcccctgctgggtggcagcaagtatggcgcctcccctctctgcctgcctttgccttttggggagcccagcaccatgggctacatggtcgctctcatcttgctcaattccctttgcttcctcatgatgaccattgcctacaccaagctctactgcaatttggacaagggagacctggagaatatttgggactgctctatggtaaaacacattgccctgttgctcttcaccaactgcatcctaaactgccctgtggctttcttgtccttctcctctttaataaaccttacatttatcagtcctgaagtaattaagtttatccttctggtggtagtcccacttcctgcatgtctcaatccccttctctacatcttgttcaatcctcactttaaggaggatctggtgagcctgagaaagcaaacctacgtctggacaagatcaaaacacccaagcttgatgtcaattaactctgatgatgtcgaaaaacagtcctgtgactcaactcaagccttggtaacctttaccagctccagcatcacttatgacctgcctcccagttccgtgccatcaccagcttatccagtgactgagagctgccatctttcctctgtggcatttgtcccatgtctctaa

SEQ ID NO: 4 - protein, 693 aa, homo sapiens
LHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSWISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 5 - DNA, 2467 bp, homo sapiens
gaaaggaggaaagaaaaaaagaggaatggaaagagacagagaaaggaaatgggagtggaaggagggaggactgctttgtaactgctaagattgcagacagaaatagcacacaaccactgtgagctgtatgcgattcagaaaccaagaccaaattttgctcactttcattaatcagttgctcagatagaaggaaatgacatctggttctgtcttcttctacatcttaatttttggaaaatatttttctcatgggggtggacaggatgtcaagtgctcccttggctatttcccctgtgggaacatcacaaagtgcttgcctcagctcctgcactgtaacggtgtggacgactgcgggaatcaggccgatgaggacaactgtggagacaacaatggatggtccatgcaatttgacaaatattttgccagttactacaaaatgacttcccaatatccttttgaggcagaaacacctgaatgtttggtcggttctgtgccagtgcaatgtctttgccaaggtctggagcttgactgtgatgaaaccaatttacgagctgttccatcggtttcttcaaatgtgactgcaatgtcacttcagtggaacttaataagaaagcttcctcctgattgcttcaagaattatcatgatcttcagaagctgtacctgcaaaacaataagattacatccatctccatctatgctttcagaggactgaatagccttactaaactgtatctcagtcataacagaataaccttcctgaagccgggtgtttttgaagatcttcacagactagaatggctgataattgaagataatcacctcagtcgaatttccccaccaacattttatggactaaattctcttattctcttagtcctgatgaataacgtcctcacccgtttacctgataaacctctctgtcaacacatgccaagactacattggctggaccttgaaggcaaccatatccataatttaagaaatttgacttttatttcctgcagtaatttaactgttttagtgatgaggaaaaacaaaattaatcacttaaatgaaaatacttttgcacctctccagaaactggatgaattggatttaggaagtaataagattgaaaatcttccaccgcttatattcaaggacctgaaggagctgtcacaattgaatctttcctataatccaatccagaaaattcaagcaaaccaatttgattatcttgtcaaactcaagtctctcagcctagaagggattgaaatttcaaatatccaacaaaggatgtttagacctcttatgaatctctctcacatatattttaagaaattccagtactgtgggtatgcaccacatgttcgcagctgtaaaccaaacactgatggaatttcatctctagagaatctcttggcaagcattattcagagagtatttgtctgggttgtatctgcagttacctgctttggaaacatttttgtcatttgcatgcgaccttatatcaggtctgagaacaagctgtatgccatgtcaatcatttctctctgctgtgccgactgcttaatgggaatatatttattcgtgatcggaggctttgacctaaagtttcgtggagaatacaataagcatgcgcagctgtggatggagagtactcattgtcagcttgtaggatctttggccattctgtccacagaagtatcagttttactgttaacatttctgacattggaaaaatacatctgcattgtctatccttttagatgtgtgagacctggaaaatgcagaacaattacagttctgattctcatttggattactggttttatagtggctttcattccattgagcaataaggaatttttcaaaaactactatggcaccaatggagtatgcttccctcttcattcagaagatacagaaagtattggagcccagatttattcagtggcaatttttcttggtattaatttggccgcatttatcatcatagttttttcctatggaagcatgttttatagtgttcatcaaagtgccataacagcaactgaaatacggaatcaagttaaaaaagagatgatccttgccaaacgttttttctttatagtatttactgatgcattatgctggatacccatttttgtagtgaaatttctttcactgcttcaggtagaaataccaggtaccataacctcttgggtagtgatttttattctgcccattaacagtgctttgaacccaattctctatactctgaccacaagaccatttaaagaaatgattcatcggttttggtataactacagacaaagaaaatctatggacagcaaaggtcagaaaacatatgctccatcattcatctgggtggaaatgtggccactgcaggagatgccacctgagttaatgaagccggaccttttcacatacccctgtgaaatgtcactgatttctcaatcaacgagactcaattcctattcatga

SEQ ID NO: 6 - protein, 757 aa, homo sapiens
MTSGSVFFYILIFGKYFSHGGGQDVKCSLGYFPCGNITKCLPQLLHCNGVDDCGNQADEDNCGDNNGWSMQFDKYFASYYKMTSQYPFEAETPECLVGSVPVQCLCQGLELDCDETNLRAVPSVSSNVTAMSLQWNLIRKLPPDCFKNYHDLQKLYLQNNKITSISIYAFRGLNSLTKLYLSHNRITFLKPGVFEDLHRLEWLIIEDNHLSRISPPTFYGLNSLILLVLMNNVLTRLPDKPLCQHMPRLHWLDLEGNHIHNLRNLTFISCSNLTVLVMRKNKINHLNENTFAPLQKLDELDLGSNKIENLPPLIFKDLKELSQLNLSYNPIQKIQANQFDYLVKLKSLSLEGIEISNIQQRMFRPLMNLSHIYFKKFQYCGYAPHVRSCKPNTDGISSLENLLASIIQRVFVWVVSAVTCFGNIFVICMRPYIRSENKLYAMSIISLCCADCLMGIYLFVIGGFDLKFRGEYNKHAQLWMESTHCQLVGSLAILSTEVSVLLLTFLTLEKYICIVYPFRCVRPGKCRTITVLILIWITGFIVAFIPLSNKEFFKNYYGTNGVCFPLHSEDTESIGAQIYSVAIFLGINLAAFIIIVFSYGSMFYSVHQSAITATEIRNQVKKEMILAKRFFFIVFTDALCWIPIFVVKFLSLLQVEIPGTITSWVVIFILPINSALNPILYTLTTRPFKEMIHRFWYNYRQRKSMDSKGQKTYAPSFIWVEMWPLQEMPPELMKPDLFTYPCEMSLISQSTRLNSYS

SEQ ID NO: 7 - DNA, 3584 bp, homo sapiens
  misc_feature 3427, 3496, 3531, 3568: n = A,T,C or G
ctgctttgtaactgctaagattgcagacagaaatagcacacaaccactgtgagctgtatgcgattcagaaaccaagaccaaattttgctcactttcattaatcagttgctcagatagaaggaaatgacatctggttctgtcttcttctacatcttaatttttggaaaatatttttctcatgggggtggacaggatgtcaagtgctcccttggctatttcccctgtgggaacatcacaaagtgcttgcctcagctcctgcactgtaacggtgtggacgactgcgggaatcaggccgatgaggacaactgtgtggtggttttgtgccagtgcatgtctttgccaggtctggagcttgactggatgaaaccatttacgagtgttccatcggtttcttcaaatgtgactgcaatgtcacttcagtggaacttaataagaaagcttcctcctgattgcttcaagaattatcatgatcttcagaagctggacctgcaaaacaataagattacatccatctccatctatgctttcagaggactgaatagccttactaaactgtatctcagtcataacagaataaccttcctgaagccgggtgtttttgaagatcttcacagactagaatggctgataattgaagataatcacctcagtcgaatttccccaccaacattttatggactaaattctcttattctcttagtcctgatgaataacgtcctcacccgtttacctgataaacctctctgtcaacacatgccaagactacattggctggaccttgaaggcaaccatatccataatttaagaaatttgacttttatttcctgcagtaatttaactgttttagtgatgaggaaaaacaaaattaatcacttaaatgaaaatacttttgcacctctccagaaactggatgaattggatttaggaagtaataagattgaaaatcttccaccgcttatattcaaggacctgaaggagctgtcacaattgaatctttcctataatccaatccagaaaattcaagcaaaccaatttgattatcttgtcaaactcaagtctctcagcctagaagggattgaaatttcaaatatccaacaaaggatgtttagacctcttatgaatctctctcacatatattttaagaaattccagtactgtgggtatgcaccacatgttcgcagctgtaaaccaaacactgatggaatttcatctctagagaatctcttggcaagcattattcagagagtatttgtctgggttgtatctgcagttacctgctttggaaacatttttgtcatttgcatgcgaccttatatcaggtctgagaacaagctgtatgccatgtcaatcatttctctctgctgtgccgactgcttaatgggaatatatttattcgtgatcggaggctttgacctaaagtttcgtggagaatacaataagcatgcgcagctgtggatggagagtactcattgtcagcttgtaggatctttggccattctgtccacagaagtatcagttttactgttaacatttctgacattggaaaaatacatctgcattgtctatccttttagatgtgtgagacctggaaaatgcagaacaattacagttctgattctcatttggattactggttttatagtggctttcattccattgagcaataaggaatttttcaaaaactactatggcaccaatggagtatgcttccctcttcattcagaagatacagaaagtattggagcccagatttattcagtggcaatttttcttggtattaatttggccgcatttatcatcatagttttttcctatggaagcatgttttatagtgttcatcaaagtgccataacagcaactgaaatacggaatcaagttaaaaaagagatgatccttgccaaacgttttttctttatagtatttactgatgcattatgctggatacccatttttgtagtgaaatttctttcactgcttcaggtagaaataccaggtaccataacctcttgggtagtgatttttattctgcccattaacagtgctttgaacccaattctctatactctgaccacaagaccatttaaagaaatgattcatcggttttggtataactacagacaaagaaaatctatggacagcaaaggtcagaaaacatatgctccatcattcatctgggtggaaatgtggccactgcaggagatgccacctgagttaatgaagccggaccttttcacatacccctgtgaaatgtcactgatttctcaatcaacgagactcaattcctattcatgactgactctgaaattcatttcttcgcagagaatactgtgggggtgcttcatgagggatttactggtatgaaaatgaataccacaaaattaatttataataatagctaagataaatattttacaaggacatgaggaaaaataaaaatgactaatgctcttacaaagggaagtaattatatcaataatgtatatatattagtagacattttgcataagaaattaagagaaatctacttcagtaacattcattcatttttctaacatgcatttattgagtacccactactatgtgcatagcattgcaatatagtcctggaagtagacagtgcagaacctttcaatctgtagatagtgtttaatgacaaaagactatacaaagtccatctgcagttcctagtttaaagtagagctttacctgtcatgtgcatcagcaagaatcataggcacttttaaataaaggtttaaagttttggaatactcagtgtatttgcatcatagaaaatgtctgactgtttgcaaaataatattctgttttaagaatccatcttacctctctttaagtttccatacacttgagagccaacacaacatatttattactaaaaagatgctttgctagaaactcaaaaacagcacttcttttggcacttcctgcccagttttctctttgctttaaatgaacatcatcatatggaattggaataggagagtatgagtacggcagagaagtggatcagaaaaactagaatgaggataaacatttacattagtggaaactcctgaaataaatccttgtattgtcagttaactgattttcaacaaggatgccaagacaaaaaggcttttcaacaaaccgtgctgttttaagaacagacctaagtggtttaattcacccactttagatgggtgaatgttatggtgtgtgaaatatctcagtaaagcagttaaaaggaaaaagagctggaatgcactgattcaggaacttaatttcaggaaggaaaggtctgtatgtacacatttcactttaagcagaaaatctttcttcaagaaatgactttactttctctttgcactgccagcacgtgagatactaactttttaactagttgttcttctctagtctctacgttattagnattttttgctttcataatgtgaaacctttaagcaggagaagaaaatgttttcagatagtttcaaatacnccaaaaatgtttgcaacacaaaaatactggaatcnaaccataatgcccttattgaatatatagttgtatagntttgttctgaaaaccc

SEQ ID NO: 8 - protein, 722 aa, homo sapiens
MTSGSVFFYILIFGKYFSHGGGQDVKCSLGYFPCGNITKCLPQLLHCNGVDDCGNQADEDNCVVVLCQCMSLPGLELDWMKPFTSVPSVSSNVTAMSLQWNLIRKLPPDCFKNYHDLQKLDLQNNKITSISIYAFRGLNSLTKLYLSHNRITFLKPGVFEDLHRLEWLIIEDNHLSRISPPTFYGLNSLILLVLMNNVLTRLPDKPLCQHMPRLHWLDLEGNHIHNLRNLTFISCSNLTVLVMRKNKINHLNENTFAPLQKLDELDLGSNKIENLPPLIFKDLKELSQLNLSYNPIQKIQANQFDYLVKLKSLSLEGIEISNIQQRMFRPLMNLSHIYFKKFQYCGYAPHVRSCKPNTDGISSLENLLASIIQRVFVWVVSAVTCFGNIFVICMRPYIRSENKLYAMSIISLCCADCLMGIYLFVIGGFDLKFRGEYNKHAQLWMESTHCQLVGSLAILSTEVSVLLLTFLTLEKYICIVYPFRCVRPGKCRTITVLILIWITGFIVAFIPLSNKEFFKNYYGTNGVCFPLHSEDTESIGAQIYSVAIFLGINLAAFIIIVFSYGSMFYSVHQSAITATEIRNQVKKEMILAKRFFFIVFTDALCWIPIFVVKFLSLLQVEIPGTITSWVVIFILPINSALNPILYTLTTRPFKEMIHRFWYNYRQRKSMDSKGQKTYAPSFIWVEMWPLQEMPPELMKPDLFTYPCEMSLISQSTRLNSYS

(19) United States
(12) Patent Application Publication
(10) Pub. No.: US 2005/0026248 A1
(43) Pub. Date: Feb. 3, 2005

(54) Novel mammalian G-protein coupled receptors having extracellular leucine rich repeat regions

(76) Inventors: Aaron J.W. Hsueh, Stanford, CA (US); Sheau Yu Hsu, Mountain View, CA (US); Shan-Guang Liang, Palo Alto, CA (US); Petrus Johannes Van Der Spek, Oss (NL)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/851,470

(22) Filed: May 21, 2004

Related U.S. Application Data
(63) Continuation of application No. 09/647,067, filed on Sep. 25, 2000.
(60) Provisional application No. 60/079,501, filed on Mar. 26, 1998.

(51) Int. Cl. C07K 14/705; C07H 21/04

(52) U.S. Cl. 435/69.1; 435/320.1; 435/325; 530/350; 536/23.5

(57) ABSTRACT

Isolated nucleotide compositions and sequences are provided for LGR4, LGR5 and LGR7 genes. The nucleic acid compositions find use in identifying homologous or related genes; in identifying endogenous ligands for these receptors; in producing compositions that modulate the expression or function of its encoded protein; for gene therapy; mapping functional regions of the protein; and in studying associated physiological pathways. In addition, modulation of the gene activity in vivo is used for prophylactic and therapeutic purposes.

### FIELD OF THE INVENTION

The field of this invention is the G-protein coupled receptor family of proteins.

### BACKGROUND

Gonadotropins (Luteinizing hormone, LH; follicle stimulating hormone, FSH; chorionic gonadotropin, CG) and thyrotropin (TSH)) are essential for the growth and differentiation of gonads and thyroid gland, respectively. These glycoprotein hormones bind specific target cell receptors on the plasma membrane to activate the cAMP-protein kinase A pathway.

The receptors for LH, FSH and TSH belong to the large G-protein-coupled, seven-trans-membrane protein family but are unique in having a large N-terminal extra-cellular (ecto-) domain containing leucine-rich repeats important for interaction with large glycoprotein ligands. Studies suggest that in these receptors, the extra-cellular leucine rich repeat region serves as a “baseball glove” which efficiently catches its corresponding large hormone ligand and optimally orients it for interaction with the seven trans-membrane-helical domain of the receptor.

Because hormones and receptors play a prominent role in a variety of physiological processes, there is continued interest in the identification of novel receptors and their ligands, as well as the genes encoding the same.

Relevant Literature

References of interest include: el Tayar, N, “Advances in the Molecular Understanding of Gonadotropins-Receptors Interactions,” Mol. Cell. Endocrinol. (Dec. 20, 1996). 125: 65-70; Bhowmick et al., “Determination of Residues Important in Hormone Binding to the Extracellular Domain of the Luteinizing Hormone/Chorionic Gonadotropin Receptor by Site-Directed Mutagenesis and Modeling,” Mol. Endocrinol. (September 1996)10:1147-1159; Thomas et al., “Mutational Analyses of the Extracellular Domain of the Full-Length Lutropin/Choriogonadotropin Receptor Suggest Leucine-Rich Repeats 1-6 are Involved in Hormone Binding,” Mol. Endocrinol. (June 1996)10:760-768; Segaloff& Ascoli, “The Gonadotropin Receptors: Insights from the Cloning of their cDNAs,” Oxf. Rev. Reprod. Biol. (1992) 14: 141-168; Braun et al., “Amino-Terminal Leucine-Rich Repeats in Gonadotropin Receptors Determine Hormone Selectivity,” EMBO J. (July 1991) 10: 1885-1890; and Segaloff et al., “Structure of the Lutropin/Choriogonadotropin Receptor,” Recent Prog. Horm. Res. (1990) 46: 261-301.

### SUMMARY OF THE INVENTION

Three novel mammalian G-protein coupled receptors having extra-cellular leucine rich repeat domains, i.e. LGR4, LGR5 and LGR7, and polypeptide compositions related thereto, as well as nucleotide compositions encoding the same, are provided. The subject proteins, polypeptide and nucleic acid compositions find use in a variety of different applications, including the identification of homologous or related genes; the production of compositions that modulate the expression or function of the subject proteins; in the identification of endogenous ligands for the subject orphan receptors; in the generation of functional binding proteins for the neutralization of the actions of endogenous ligands; in gene therapy; in mapping functional regions of the protein; and in studying associated physiological pathways. In addition, modulation of the gene activity in vivo is used for prophylactic and therapeutic purposes, and the like.

### BRIEF DESCRIPTION OF THE FIGURES

FIG. 1 provides the nucleotide and amino acid sequence for human LGR4.

FIG. 2 provides the nucleotide and amino acid sequence for human LGR5.

FIG. 3 provides the nucleotide and amino acid sequence for human LGR7, long form.

FIG. 4 provides the nucleotide and amino acid sequence for human LGR7, short form.

FIG. 5 provides an alignment comparison of the long and short forms of LGR7.

FIG. 6 provides a comparison of deduced amino acid sequence of LGR4 and 5 cDNAs and those encoding FSH and LH receptors.

### DESCRIPTION OF THE SPECIFIC EMBODIMENTS

Novel mammalian G-protein coupled receptors having extra-cellular leucine rich repeat regions (i.e. LGR4, LGR5 and LGR7) and polypeptide compositions related thereto, as well as nucleic acid compositions encoding the same, are provided. The subject polypeptide and/or nucleic acid compositions find use in a variety of different applications, including the identification of homologous or related genes; for the identification of endogenous ligands for these novel receptors; the production of compositions that modulate the expression or function of the receptors; for gene therapy; for mapping functional regions of the receptors; in studying associated physiological pathways; for in vivo prophylactic and therapeutic purposes; as immunogens for producing antibodies; in screening for biologically active agents; and the like.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

### Characterization of LGR4, LGR5 AND LGR7

LGR4, LGR5 and LGR7 are novel mammalian receptors of the G-protein coupled, seven trans-membrane family of proteins, specifically the subfamily of G-protein coupled seven trans-membrane proteins which are characterized by the presence of extra-cellular leucine rich repeat regions. As such, these proteins have trans-membrane segments and extra-cellular regions similar to those found in the known LH, FSH, and TSH receptors. In other words, these proteins have both a G-protein coupled seven trans-membrane region and a leucine rich repeat extra-cellular domain. The N-terminal extra-cellular domains of these proteins also show high homology with Drosophila Slit and Toll proteins having leucine rich repeats. These proteins are expressed in diverse tissues.

The human LGR4 gene has a nucleotide sequence as shown in SEQ ID NO:01. The human LGR4 gene product has an amino acid sequence as shown in SEQ ID NO:02. LGR4 is expressed in a plurality of different tissue types, including ovary, testis, adrenal, placenta, liver, kidney and intestine.

The human LGR5 gene has a nucleotide sequence as shown in SEQ ID NO:03. The LGR5 gene product has an amino acid sequence as shown in SEQ ID NO:04. LGR5 has been found to be mainly expressed in muscle, placenta and spinal cord tissue.

The human LGR7 gene encodes multiple splicing variants, each of which contains a multitude of cysteine-rich low density lipoprotein (LDL) binding motifs at the N-terminus in addition to the luecine rich repeat region. The longer forms of LGR-7 have a higher similarity than shorter forms of LGR-7 to snail LGR in the trans-membrane domain and the N-terminal LDL binding domain. The overall structure of both the long and short forms of LGR-7 is similar to that of the LH receptor. The human LGR7 short form gene has a nucleotide sequence as shown in SEQ ID NO:05. The LGR7 short form gene product has an amino acid sequence as shown in SEQ ID NO:06. The human LGR7 long form gene has a nucleotide sequence as shown in SEQ ID NO:07. The LGR7 long form gene product has an amino acid sequence as shown in SEQ ID NO:08. LGR7 is expressed in multiple tissues, including testis, ovary, prostate, intestine and colon.

### Identification of LGR4, LGR5 and LGR7 Sequences

Homologs of LGR4, LGR5 and LGR 7 are identified by any of a number of methods. A fragment of the provided cDNA may be used as a hybridization probe against a cDNA library from the target organism of interest, where low stringency conditions are used. The probe may be a large fragment, or one or more short degenerate primers.

Nucleic acids having sequence similarity are detected by hybridization under low stringency conditions, for example, at 50° C. and 6×SSC (0.9 M sodium chloride/0.09 M sodium citrate) and remain bound when subjected to washing at 55° C. in 1×SSC (0.15 M sodium chloride/0.015 M sodium citrate). Sequence identity may be determined by hybridization under stringent conditions, for example, at 50° C. or higher and 0.1 ×SSC (15 mM sodium chloride/01.5 mM sodium citrate). Nucleic acids having a region of substantial identity to the provided LGR4, LGR5 and/or LGR7 sequences, e.g. allelic variants, genetically altered versions of the gene, etc., bind to the provided sequences under stringent hybridization conditions. By using probes, particularly labeled probes of DNA sequences, one can isolate homologous or related genes. The source of homologous genes may be any species, e.g., primate species, particularly human; rodents, such as rats and mice; canines; felines; bovines; ovines; equines; yeast; nematodes; etc.

Between mammalian species, e.g., human and mouse, homologs have substantial sequence similarity, e.g. at least 75% sequence identity, usually at least 90%, more usually at least 95% between nucleotide sequences. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul el al. (1990), J. Mol. Biol. 215:403-10. Unless specified otherwise, all sequence analysis numbers provided herein are as determined with the BLAST program using default settings. The sequences provided herein are essential for recognizing LGR4, LGR5 and LGR7-related and homologous proteins in database searches.

### LGR4, LGR5 and LGR7 Nucleic Acid Compositions

Nucleic acids encoding LGR4, LGR5 and LGR7 may be cDNA or genomic DNA or a fragment thereof. The terms “LGR4 gene,” “LGR5 gene ” “and “LGR7 gene” shall be intended to mean the open reading frame encoding specific LGR4, LGR5 and LGR7 polypeptides, and LGR4, LGR5 and LGR7 introns, as well as adjacent 5′ and 3′ non-coding nucleotide sequences involved in the regulation of expression, up to about 20 kb beyond the coding region, but possibly further in either direction. The gene may be introduced into an appropriate vector for extra-chromosomal maintenance or for integration into a host genome.

The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, removed by nuclear RNA splicing, to create a continuous open reading frame encoding an LGR4, LGR5 and LGR7 protein.

A genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The sequence of the 5′ flanking region may be utilized for promoter elements, including enhancer binding sites, that provide for developmental regulation in tissues where LGR4, LGR5 and/or LGR7 is expressed. The tissue specific expression is useful for determining the pattern of expression, and for providing promoters that mimic the native pattern of expression. Naturally occurring polymorphisms in the promoter region are useful for determining natural variations in expression, particularly those that may be associated with disease.

Alternatively, mutations may be introduced into the promoter region to determine the effect of altering expression in experimentally defined systems. Methods for the identification of specific DNA motifs involved in the binding of transcriptional factors are known in the art, e.g. sequence similarity to known binding motifs, gel retardation studies, etc. For examples, see Blackwell et al. (1995), Mol. Med. 1: 194-205; Mortlock et al. (1996), Genome Res. 6:327-33; and Joulin and Richard-Foy (1995), Eur. J. Biochem. 232:620-626.

The regulatory sequences may be used to identify cis acting sequences required for transcriptional or translational regulation of LGR4, LGR5 and/or LGR 7 expression, especially in different tissues or stages of development, and to identify cis acting sequences and trans-acting factors that regulate or mediate LGR4, LGR and/or LGR7 expression. Such transcription or translational control regions may be operably linked to an LGR4, LGR5 or LGR 7 gene in order to promote expression of wild type or altered LGR4, LGR5 or LGR7 or other proteins of interest in cultured cells, or in embryonic, fetal or adult tissues, and for gene therapy.

The nucleic acid compositions of the subject invention may encode all or a part of the subject polypeptides. Double or single stranded fragments may be obtained of the DNA sequence by chemically synthesizing oligonucleotides in accordance with conventional methods, by restriction enzyme digestion, by PCR amplification, etc. For the most part, DNA fragments will be of at least 15 nt, usually at least 18 nt or 25 nt, and may be at least about 50 nt. Such small DNA fragments are useful as primers for PCR, hybridization screening probes, etc. Larger DNA fragments, i.e. greater than 100 nt are useful for production of the encoded polypeptide. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

The LGR4, LGR and LGR7 genes are isolated and obtained in substantial purity, generally as other than an intact chromosome. Usually, the DNA will be obtained substantially free of other nucleic acid sequences that do not include an LGR4, LGR5 or LGR 7 sequence or fragment thereof, generally being at least about 50%, usually at least about 90% pure and are typically “recombinant”, i.e. flanked by one or more nucleotides with which it is not normally associated on a naturally occurring chromosome.

The DNA may also be used to identify expression of the gene in a biological specimen. The manner in which one probes cells for the presence of particular nucleotide sequences, as genomic DNA or RNA, is well established in the literature and does not require elaboration here. DNA or mRNA is isolated from a cell sample. The mRNA may be amplified by RT-PCR, using reverse transcriptase to form a complementary DNA strand, followed by polymerase chain reaction amplification using primers specific for the subject DNA sequences. Alternatively, the mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of LGR4, LGR5 and/or LGR7 gene expression in the sample.

The sequence of an LGR4, LGR5 or LGR7 gene, including flanking promoter regions and coding regions, may be mutated in various ways known in the art to generate targeted changes in promoter strength, sequence of the encoded protein, etc. The DNA sequence or protein product of such a mutation will usually be substantially similar to the sequences provided herein, i.e. will differ by at least one nucleotide or amino acid, respectively, and may differ by at least two but not more than about ten nucleotides or amino acids. The sequence changes may be substitutions, insertions, deletions, or a combination thereof. Deletions may further include larger changes, such as deletions of a domain or exon. Other modifications of interest include epitope tagging, e.g. with the FLAG system, HA, etc. For studies of subcellular localization, fusion proteins with green fluorescent proteins (GFP) may be used.

Techniques for in vitro mutagenesis of cloned genes are known. Examples of protocols for site specific mutagenesis may be found in Gustin et al. (1993), Biotechniques 14:22; Barany (1985), Gene 37:111-23; Colicelli et al. (1985), Mol. Gen. Genet 199:537-9; and Prentki et al. (1984), Gene 29:303-13. Methods for site specific mutagenesis can be found in Sambrook et al., Molecular Cloning: A Laboratory Manual, CSH Press 1989, pp. 15.3-15.108; Weiner et al. (1993), Gene 126:3541; Sayers et al. (1992), Biotechniques 13:592-6; Jones and Winistorfer (1992), Biotechniques 12:528-30; Barton et al. (1990), Nucleic Acids Res 18:7349-55; Marotti and Tomich (1989), Gene Anal. Tech. 6:67-70; and Zhu (1989), Anal Biochem 177:120-4. Such mutated genes may be used to study structure-function relationships of LGR4, LGR5 and/or LGR7, or to alter properties of the protein that affect its function or regulation.

### LGR4, LGR5 and LGR7 Polypeptides

Also provided by the subject invention are LGR4, LGR5 and LGR7 polypeptide compositions. The term polyeptide composition as used herein refers to both the full length proteins as well as portions or fragments thereof. Also included in this term are variations of the naturally-occurring proteins, where such variations are homologous or substantially similar to the naturally occurring protein, be the naturally occurring protein the human protein, mouse protein, or protein from some other species which naturally expresses an LGR4, LGR5 or LGR7 protein, usually a mammalian species. A candidate homologous protein is substantially similar to an LGR4, LGR5 or LGR7 protein of the subject invention, and therefore is an LGR4, LGR5 or LGR7 protein of the subject invention, if the candidate protein has a sequence that has at least about 80%, usually at least about 90% and more usually at least about 98% sequence identity with an LGR4, LGR5 or LGR7 protein, as measured by BLAST, supra. In the following description of the subject invention, the term “LGR4, LGR5 or LGR7-protein” is used to refer not only to the human LGR4, LGR5 or LGR7 protein, but also to homologs thereof expressed in non-human species, e.g. murine, rat and other mammalian species.

The subject gene may be employed for producing all or portions of LGR4, LGR5 and LGR7 polypeptides. By “LGR4 polypeptide/protein”, “LGR5 polypeptide/protein,” and “LGR7 polypeptide/protein” is meant an amino acid sequence encoded by an open reading frame (ORF) of LGR4, LGR5 and LGR 7 genes, including the full-length native polypeptide and fragments thereof, particularly biologically active fragments and/or fragments corresponding to functional domains, e.g. extra-cellular regions; and including fusions of the subject polypeptides to other proteins or parts thereof, e.g. chimeric proteins. For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to an LGR4, LGR5 or LGR 7 gene, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, and up to the complete open reading frame of the gene. After introduction of the DNA, the cells containing the construct may be selected by means of a selectable marker, the cells expanded and then used for expression.

LGR4, LGR5 or LGR7 polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells. In some situations, it is desirable to express the LGR4, LGR5 or LGR 7 gene in eukaryotic cells, where the LGR4, LGR5 or LGR7 protein will benefit from native folding and post-translational modifications. Small peptides can also be synthesized in the laboratory. Polypeptides that are subsets of the complete LGR4, LGR5 or LGR7 sequence may be used to identify and investigate parts of the protein important for function or to raise antibodies directed against these regions.

For production of the extracellular domain of the LGR4, LGR5 or LGR7 receptor, the anchored receptor approach as described in Osuga et al, Mol. Endocrinol. (1997) 11: 1659-1668 may be employed. Likewise, the chimeric receptor approach described in Kudo et al, J. Biol. Chem. (1996) 271; 22470-22478 may be used.

Such peptides find use in the identification of endogenous ligands and in drug screening for agonists and atangonists using methods described in Osuga, supra. Solubilized extracellular domains find use as therapeutic agents, e.g. in the neutralization of the action of endogenous ligands.

With the availability of the protein or fragments thereof in large amounts, by employing an expression host, the protein may be isolated and purified in accordance with conventional ways. A lysate may be prepared of the expression host and the lysate purified using HPLC, exclusion chromatography, gel electrophoresis, affinity chromatography, or other purification technique. The purified protein will generally be at least about 80% pure, preferably at least about 90% pure, and may be up to and including 100% pure. Pure is intended to mean free of other proteins, as well as cellular debris.

The expressed LGR4, LGR5 and LGR7 polypeptides are useful for the production of antibodies, where short fragments provide for antibodies specific for the particular polypeptide, and larger fragments or the entire protein allow for the production of antibodies over the surface of the polypeptide. Antibodies may be raised to the wild-type or variant forms of LGR4, LGR5 or LGR7. Antibodies may be raised to isolated peptides corresponding to these domains, or to the native protein.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. Both polyclonal and monoclonal antibodies may be produced. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage “display” libraries, usually in conjunction with in vitro affinity maturation.

### Diagnostic Uses

The subject nucleic acid and/or polypeptide compositions may be used to analyze a patient sample for the presence of polymorphisms associated with a disease state or genetic predisposition to a disease state. Biochemical studies may be performed to determine whether a sequence polymorphism in an LGR4, LGR or LGR7 coding region or control regions is associated with disease. Disease associated polymorphisms may include deletion or truncation of the gene, mutations that alter expression level, that affect the activity of the protein, and the like.

Changes in the promoter or enhancer sequence that may affect expression levels of LGR4, LGR5 or LGR 7 can be compared to expression levels of the normal allele by various methods known in the art. Methods for determining promoter or enhancer strength include quantitation of the expressed natural protein; insertion of the variant control element into a vector with a reporter gene such as β-galactosidase, luciferase, chloramphenicol acetyltransferase, etc., that provides for convenient quantitation; and the like.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express LGR4, LGR5 or LGR7 may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki, et al. (1985), Science 239:487, and a review of techniques may be found in Sambrook, et al. Molecular Cloning: A Laboratory Manual, CSH Press 1989, pp.14.2-14.33. Alternatively, various methods are known in the art that utilize oligonucleotide ligation as a means of detecting polymorphisms, for examples see Riley et al (1990), Nucl. Acids Res. 18:2887-2890; and Delahunty et al. (1996), Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′, 4′, 7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product. The sample nucleic acid, e.g. amplified or cloned fragment, is analyzed by one of a number of methods known in the art. The nucleic acid may be sequenced by dideoxy or other methods, and the sequence of bases compared to a wild-type LGR4, LGR5 or LGR 7 sequence. Hybridization with the variant sequence may also be used to determine its presence, by Southern blots, dot blots, etc. The hybridization pattern of a control and variant sequence to an array of oligonucleotide probes immobilized on a solid support, as described in U.S. Pat. No. 5,445,934, or in WO 95/35505 (the disclosures of which are herein incorporated by reference), may also be used as a means of detecting the presence of variant sequences. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations in LGR4, LGR5 or LGR7 may be based on the functional or antigenic characteristics of the protein. Protein truncation assays are useful in detecting deletions that may affect the biological activity of the protein. Various immunoassays designed to detect polymorphisms in LGR4, LGR5 or LGR7 proteins may be used in screening. Where many diverse genetic mutations lead to a particular disease phenotype, functional protein assays have proven to be effective screening tools. The activity of the encoded LGR4, LGR5 or LGR7 protein may be determined by comparison with the wild-type protein.

Antibodies specific for LGR4, LGR5 or LGR7 proteins may be used in staining or in immunoassays. Samples, as used herein, include biological fluids such as semen, blood, cerebrospinal fluid, tears, saliva, lymph, dialysis fluid and the like; organ or tissue culture derived fluids; and fluids extracted from physiological tissues. Also included in the term are derivatives and fractions of such fluids. The cells may be dissociated, in the case of solid tissues, or tissue sections may be analyzed. Alternatively a lysate of the cells may be prepared. Diagnosis may be performed by a number of methods to determine the absence or presence or altered amounts of normal or abnormal LGR4, LGR5 or LGR7 in patient cells. For example, detection may utilize staining of cells or histological sections, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc. Diagnostic screening may also be performed for polymorphisms that are genetically linked to a disease predisposition, particularly through the use of microsatellite markers or single nucleotide polymorphisms. Frequently the microsatellite polymorphism itself is not phenotypically expressed, but is linked to sequences that result in a disease predisposition. However, in some cases the microsatellite sequence itself may affect gene expression. Microsatellite linkage analysis may be performed alone, or in combination with direct detection of polymorphisms, as described above. The use of microsatellite markers for genotyping is well documented. For examples, see Mansfield et al. (1994), Genomics 24:225-233; Ziegle et al. (1992), Genomics 14:1026-1031; Dib et al., supra.

### Modulation of LGR4, LGR5 and LGR7 Gene Expression

The LGR4, LGR5 or LGR7 genes, gene fragments, or the LGR4, LGR5 or LGR7 protein or protein fragments, are useful in gene therapy to treat disorders associated with LGR4, LGR5 or LGR7 defects. Expression vectors may be used to introduce the LGR4, LGR5 or LGR7 gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or LGR4, LGR5 or LGR7 protein may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the LGR4, LGR5 or LGR7 DNA, then bombarded into skin cells.

Antisense molecules can be used to down-regulate expression of LGR4, LGR5, or LGR7 in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

### Genetically Altered Cell or Animal Models for LGR4, LGR5 and LGR7 Function

The subject nucleic acids can be used to generate transgenic, non-human animals or site specific gene modifications in cell lines. Transgenic animals may be made through homologous recombination, where the normal LGR4, LGR5 or LGR7 locus is altered. Alternatively, a nucleic acid construct is randomly integrated into the genome. Vectors for stable integration include plasmids, retroviruses and other animal viruses, YACs, and the like.

The modified cells or animals are useful in the study of LGR4, LGR5 and/or LGR7 function and regulation. For example, a series of small deletions and/or substitutions may be made in the host's native LGR4, LGR5 or LGR7 gene to determine the role of different exons. Of interest is the use of LGR4, LGR5 or LGR7 to construct transgenic animal models for disease states. Specific constructs of interest include anti-sense LGR4, LGR5 or LGR 7, which will block LGR4, LGR5 or LGR7 expression, expression of dominant negative LGR4, LGR5 or LGR 7 mutations, and over-expression of LGR4, LGR5 or LGR 7 genes. Where an LGR4, LGR5 or LGR7 sequence is introduced, the introduced sequence may be either a complete or partial sequence of an LGR4, LGR5 or LGR 7 gene native to the host, or may be a complete or partial LGR4, LGR5 or LGR7 sequence that is exogenous to the host animal, e.g., a human LGR4, LGR5 or LGR7 sequence. A detectable marker, such as lac Z may be introduced into the LGR4, LGR5 or LGR 7 locus, where upregulation of LGR4, LGR5 or LGR 7 expression will result in an easily detected change in phenotype.

One may also provide for expression of the LGR4, LGR5 or LGR7 gene or variants thereof in cells or tissues where it is not normally expressed, at levels not normally present in such cells or tissues, or at abnormal times of development. By providing expression of LGR4, LGR5 or LGR7 protein in cells in which it is not normally produced, one can induce changes in cell behavior, e.g. through LGR4, LGR5 or LGR7 mediated activity.

DNA constructs for homologous recombination will comprise at least a portion of the LGR4, LGR5 or LGR7 gene, which may or may not be native to the species of the host animal, wherein the gene has the desired genetic modification(s), and includes regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990), Meth. Enzymol. 185:527-537.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals may be any non-human mammal, such as laboratory animals, domestic animals, etc. The transgenic animals may be used in functional studies, drug screening, etc., e.g. to determine the effect of a candidate drug on LGR4, LGR5 or LGR 7 or related gene activation etc.

### In Vitro Models for LGR4, LGR5 or LGR7 Function

The availability of a number of components in the G-protein coupled receptor family, as previously described, allows in vitro reconstruction of the processes or systems in which members of this family operate. Two or more of the components, such as the isolated receptor and a potential ligand therefore, may be combined in vitro, and the behavior assessed in terms of activation of transcription of specific target sequences; modification of protein components, e.g. proteolytic processing, phosphorylation, methylation, etc.; ability of different protein components to bind to each other. The components may be modified by sequence deletion, substitution, etc. to determine the functional role of specific domains.

Drug screening may be performed using an in vitro model, a genetically altered cell or animal, purified LGR4, LGR5 or LGR7 protein, as well as fragments or portions thereof, e.g. solubilized extra-cellular domain or chimeric receptor proteins comprising the LGR4, LGR5 or LGR7 extra-cellular domain. One can identify ligands or substrates that bind to and modulate the action of LGR4, LGR5 or LGR7. Areas of investigation include the development of agents that beneficially counter abnormalities related to LGR4, LGR5 or LGR7 and the use of such agents in the therapy.

Drug screening identifies agents that modulate the activity of LGR4, LGR5 or LGR7 function in abnormal cells. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions, such as GTP binding, etc.

The term “agent” as used herein describes any molecule, e.g. protein or pharmaceutical, with the capability of altering or mimicking the physiological function of LGR4, LGR5 or LGR7. Generally a plurality of assay mixtures are run in parallel with different agent concentrations to obtain a differential response to the various concentrations. Typically, one of these concentrations serves as a negative control, i.e. at zero concentration or below the level of detection.

In some embodiments, candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Of particular interest in certain embodiments are peptidic agents based on LGR4, LGR5 or LGR7, e.g. solubilized extra-cellular domain or chimeric receptor proteins comprising the LGR4, LGR5 or LGR7 extra-cellular domain, where such agents neutralize the activity of endogenous LGR4, LGR5 or LGR7 ligands, e.g. hormones.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc., that are used to facilitate optimal protein-protein binding and/or reduce non-specific or background interactions. Reagents that improve the efficiency of the assay, such as protease inhibitors, nuclease inhibitors, anti-microbial agents, etc., may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Other assays of interest detect agents that mimic LGR4, LGR5 or LGR7 function. For example, an expression construct comprising an LGR4, LGR5 or LGR7 gene may be introduced into a cell line under conditions that allow expression. The level of LGR4, LGR5 or LGR7 activity is determined by a functional assay, as previously described. In one screening assay, the ability of candidate agents to inhibit or enhance LGR4, LGR5 or LGR7 function is determined. Alternatively, candidate agents are added to a cell that lacks functional LGR4, LGR5 or LGR7, and screened for the ability to reproduce LGR4, LGR5 or LGR7 activity in a functional assay.

The compounds having the desired pharmacological activity may be administered in a physiologically acceptable carrier to a host for treatment, etc. The compounds may also be used to enhance LGR4, LGR5 or LGR 7 function. The inhibitory agents may be administered in a variety of ways, orally, topically, parenterally e.g. subcutaneously, intraperitoneally, by viral infection, intravascularly, etc. Topical treatments are of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Identification of LGR4 and LGR5

Human sequences related to the sea anemone and Drosophila glycoprotein hormone receptors were identified from the expression sequence tag database (dbEST) at the National Center for Biotechnology Information by using the BLAST server with the BLOSUM62 protein comparison matrix (Altschul SF et al, Nucleic Acids Res (1997) 25:3389-3402). Human ESTs showing high homology to two non-overlapping regions of the gonadotropin receptors were identified. Clones AA312798 and AA298810 were found to encode transmembrane four to five of the putative receptor LGR4 whereas AA460529 and AA424098 encode transmembrane two to three of the putative receptor LGR5. Using these ESTs to further search the GenBank EST division database, overlapping EST sequences were aligned to obtain the longest open reading frame (ORF) for these receptors.

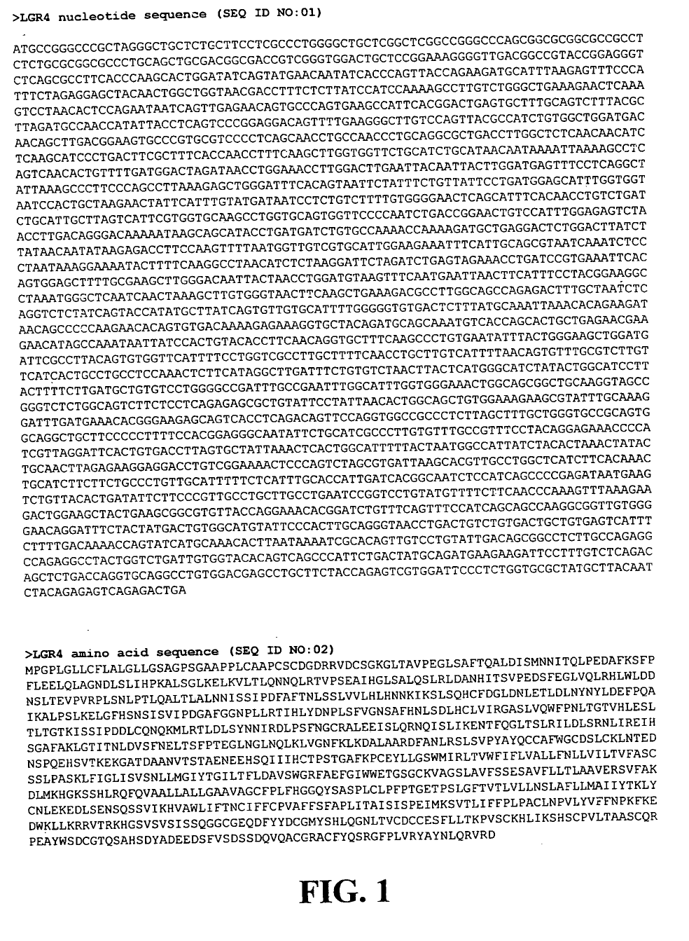

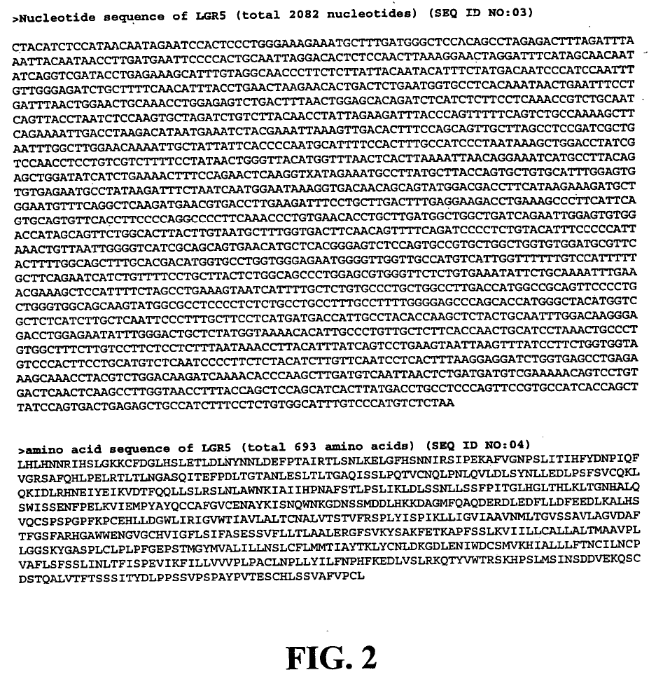

Based on the longest human ORF, specific primers were designed for PCR amplification of LGR4 and LGR5 cDNA fragments from rat ovary and human placenta, respectively. After hybridization with labeled EST clones and confirmation of DNA sequences by dideoxy DNA sequencing, specific receptor fragments isolated were used to design primers to prepare sub-cDNA libraries enriched with specific receptor cDNAs. For 5′ extension, reverse transcription was performed using rat ovarian and human placenta mRNA preparations and receptor-specific primers. Following second strand synthesis, the enriched cDNA pool was tailed at 5′-ends with specific adaptor sequences to allow further PCR amplification. For 3′ extension, rat ovarian or human placenta mRNAs were reversed transcribed using oligo-dT, followed by second strand synthesis using receptor-specific primers and adaptor tailing. These mini-libraries were further used as templates for PCR amplification of upstream or downstream cDNAs specific for each receptor using internal primers. PCR products with a strong hybridization signal to each receptor cDNA fragment were subcloned into the pUC18 or pcDNA3 vectors. After screening of these sublibraries based on colony hybridization using specific receptor probes, clones with 5′- or 3′-sequences of the putative receptors were identified and isolated for DNA sequencing. As needed, the procedure was repeated up to three times to generate cDNAs encoding the complete ORF of each putative receptor for sequence analysis and for the expression of receptor proteins in eukaryotic cells. The entire coding sequences of each gene were also amplified with specific primers flanking the entire ORF in independent experiments. At least three independent PCR clones were sequenced to verify the authenticity of coding sequences. The nucleotide sequence of LGR4, as well as the amino acid sequence of the product encoded by the ORF thereof, is provided in FIG. 1. The nucleotide sequence of LGR5, as well as the amino acid sequence of the product encoded by the ORF thereof, is provided in FIG. 2.

### Example 2

### Comparison of Deduced Amino Acid Sequence of LGR4 and 5 cDNAs and Those Encoding FSH and LH Receptors.

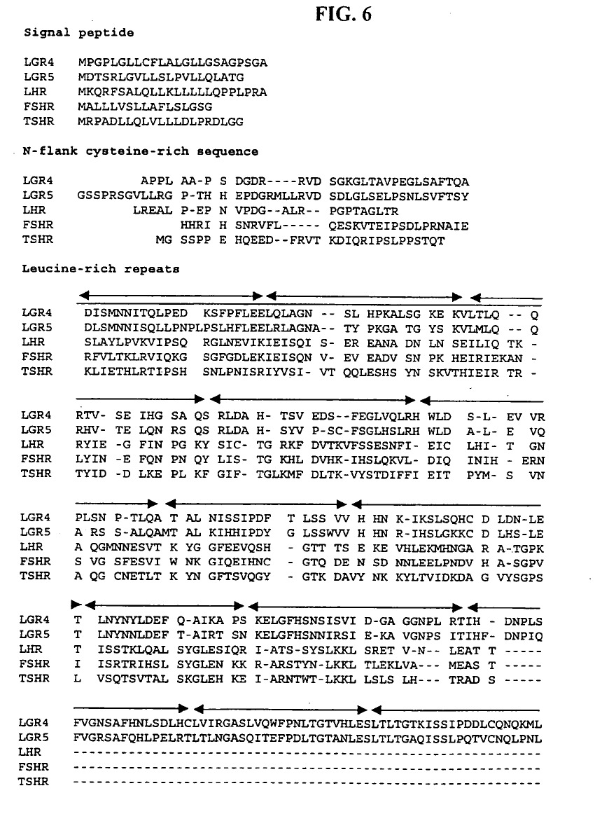

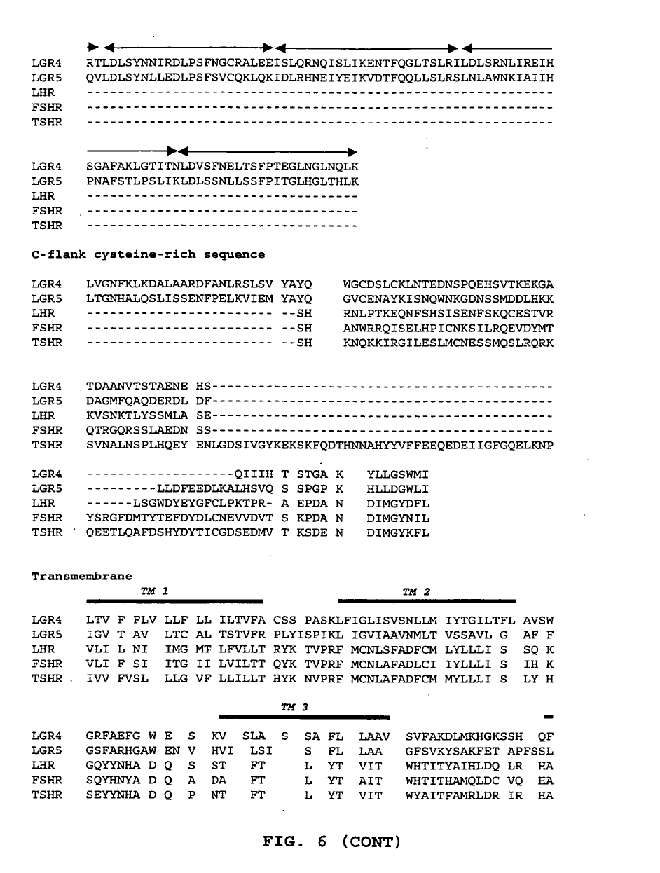

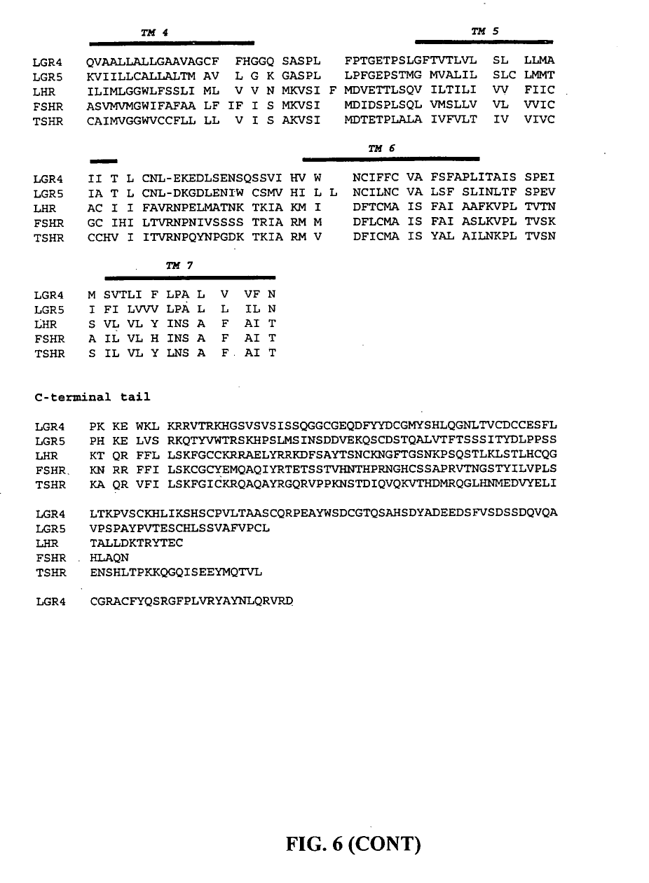

Sequence alignment of LGR4 and LGR5 with known human glycoprotein hormone receptors was performed and the results are shown in FIG. 6. Shaded residues are identical in at least two of the four receptor proteins shown.

### Example 3

### Expression Pattern of LGR4 and 5 mRNA Transcripts in Different Tissues.

For northern blot analysis, poly (A)+-selected RNA from different human tissues was hybridized with a ³²P-labeled cDNA probes. After washing, the blots were exposed to X-ray films at −70° C. for five days. Subsequent hybridization with a beta-actin cDNA probe was performed to estimate nucleic acid loading (8 h exposure). LGR4 was shown to be expressed in placenta, ovary, testis, adrenal, spinal cord, thyroid, stomach, trachea, heart, pancreas, kidney, prostate and spleen while LGR5 was shown to be expressed in the skeletal muscle, placenta, spinal cord, brain, adrenal, colon, stomach, ovary and bone marrow.

### Example 4

### Chromosomal Localization of LGR4 and 5 in Human.

Using genomic fragments of LGR4 (>100 Kb) and LGR5 (>100 Kb) as probes, chromosomal localization of these genes were detected using the FISH method to banded DNA in chromosomal 5q34-35.1 and 12q15, respectively.

### Example 5

### Identification of LGR7.

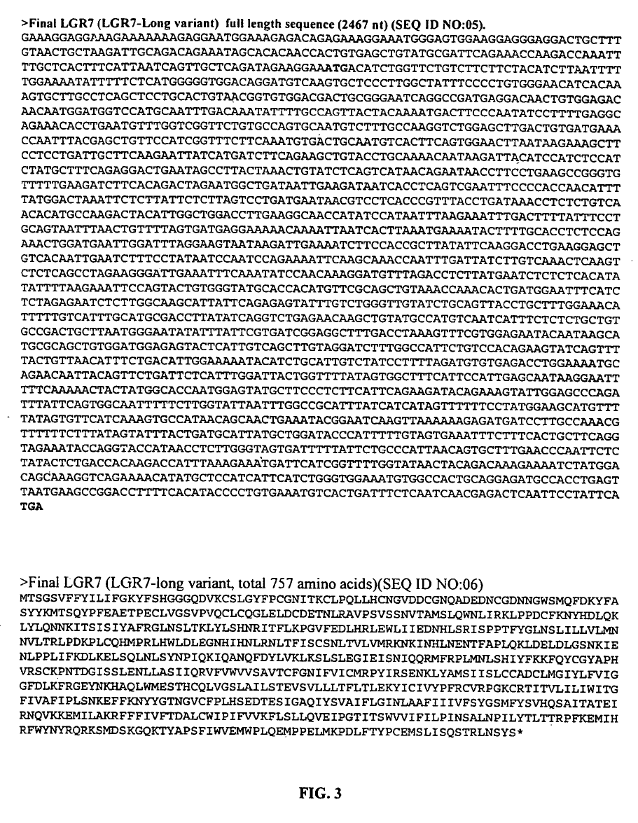

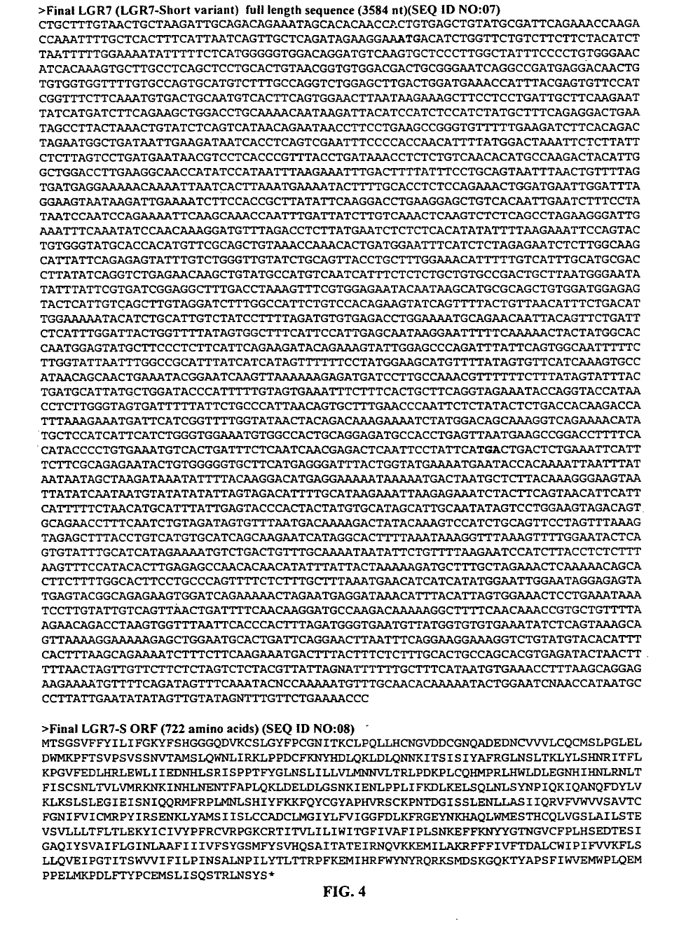

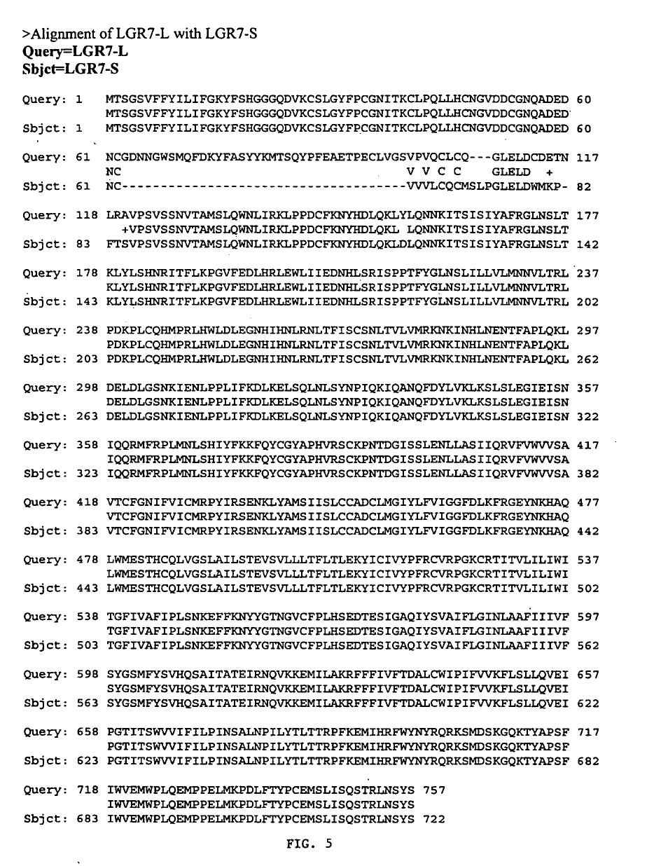

Analysis of EST databases has revealed a novel LGR closely related to a G protein-coupled receptor from pond snail (Lymnaea stagnalis, accession no. 481946). Because the snail G-protein coupled receptor shared the leucine-rich repeat ectodomain and seven transmembrane region characteristics of mammalian LGRs, the novel EST sequence could encode either a homologue of snail receptor or a novel mammalian LGR. For the isolation of LGR7 cDNA, a Clontech Marathon-ready testis cDNA pool was used as the template for 5′ and 3′ RACE with adapter and gene-specific primers. Sequence analysis of the RACE products showed that LGR7 gene encode at least two splicing variants differ at the N-terminus. The nucleotide sequence of the long variant, as well as the amino acid sequence of the product encoded by the ORF thereof, is provided in FIG. 3; while the nucleotide sequence of the short variant, as well as the amino acid sequence of the ORF thereof, is provided in FIG. 4. Both variants contain a classical C-terminal 7-transmembrane region and a leucine-rich repeat ectodomain flanked by cysteine rich regions found in other mammalian LGRs. The long form LGR7 contains extra 35 amino acids in the N-terminal cysteine rich region as compared to the short form LGR7. Of interest, analysis of the LGR7 ORF from either variant showed that its tertiary structure resembles that of mammalian LGRs instead of the snail receptor, which shares the greatest identity in the transmembrane region. These findings suggest that LGR7 and snail receptor diverged early during evolution and LGR7 perhaps adopted new function in higher organisms.

Based on the LGR7 cDNA sequence, we further identified a human genomic DNA fragment (AQ053279) in the genomic survey sequence division of GenBank that contains part of the LGR7 gene. The authenticity of this genomic clone was confirmed by Southern blot hybridization and the genomic clone was used as the probe to identify the chromosomal localization for LGR7 gene.

It is evident from the above discussion and results that three novel mammalian G-protein coupled receptors, as well as a nucleic acids encoding the same, are provided by the subject invention. The inventions described above find use in a variety of applications, including research and therapeutic applications.

All publications and patent applications cited in this specification are herein incorporated by reference as if each individual publication or patent application were specifically and individually indicated to be incorporated by reference. The publications discussed herein are provided solely for their disclosure prior to the filing date of the present application. Nothing herein is to be construed as an admission that the invention is not entitled to antedate such a disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. An isolated nucleic acid encoding a mammalian protein selected from the group consisting of LGR4, LGR5 or LGR7.

2. An isolated nucleic acid according to claim 1, wherein said mammalian protein has the amino acid sequence of SEQ ID NO:2, SEQ ID NO:04, SEQ ID NO:06 or SEQ ID NO:08.

3. An isolated nucleic acid according to claim 1, wherein said mammalian protein has an amino acid sequence that is substantially identical to the amino acid sequence of SEQ ID NO:2, SEQ ID NO:04, SEQ ID NO:06 or SEQ ID NO:08.

4. An isolated nucleic acid according to claim 1, wherein the nucleotide sequence of said nucleic acid has the sequence selected from the group consisting of: (a) SEQ ID NO:1 or the complementary sequence thereof; (b) SEQ ID NO:03 or the complementary sequence thereof; (c) SEQ ID NO:05 or the complementary sequence thereof; and (d) SEQ ID NO:07 or the complementary sequence thereof.

5. An isolated nucleic acid comprising at least 18 contiguous nucleotides of the sequence selected from the group consisting of: (a) SEQ ID NO:1 or the complementary sequence thereof; (b) SEQ ID NO:03-or the complementary sequence thereof; (c) SEQ ID NO:05 or the complementary sequence thereof, and (d) SEQ ID NO:07 or the complementary sequence thereof.

6. An isolated nucleic acid comprising at least 50 contiguous nucleotides of the sequence selected from the group consisting of: (a) SEQ ID NO:1 or the complementary sequence thereof; (b) SEQ ID NO:03 or the complementary sequence thereof; (c) SEQ ID NO:05 or the complementary sequence thereof; and (d) SEQ ID NO:07 or the complementary sequence thereof.

7. An isolated nucleic acid that hybridizes under stringent conditions to a nucleic acid having the nucleotide sequence selected from the group consisting of: (a) SEQ ID NO:1 or the complementary sequence thereof; (b) SEQ ID NO:03 or the complementary sequence thereof; (c) SEQ ID NO:05 or the complementary sequence thereof; and (d) SEQ ID NO:07 or the complementary sequence thereof.

8. An expression cassette comprising a transcriptional initiation region functional in an expression host, a nucleic acid having a sequence of the isolated nucleic acid according to claim 1 under the transcriptional regulation of said transcriptional initiation region, and a transcriptional termination region functional in said expression host.

9. A cell comprising an expression cassette according to claim 8 as part of an extrachromosomal element or integrated into the genome of a host cell as a result of introduction of said expression cassette into said host cell, and the cellular progeny of said host cell.

10. A method for producing a mammalian protein selected from the group consisting of LGR4, LGR5 and LGR7, said method comprising:
growing a cell according to claim 9, whereby said mammalian protein is expressed; and
isolating said protein substantially free of other proteins.

11. A purified polypeptide composition comprising at least 50 weight % of the protein present as a mammalian protein selected from the group consisting of LGR4, LGR5 and LGR7, or a fragment thereof.

12. An antibody binding specifically to a mammalian protein selected from the group consisting of LGR4, LGR5 and LGR7.

13. The antibody of claim 12, wherein said antibody is a monoclonal antibody.

14. A non-human transgenic animal model for LGR4, LGR5 or LGR7 gene function, wherein said transgenic animal comprises an introduced alteration in an LGR4, LGR5 or LGR7 gene.

15. The animal model of claim 14, wherein said animal is heterozygous for said introduced alteration.

16. The animal model of claim 14, wherein said animal is homozygous for said introduced alteration.

17. The animal model of claim 14, wherein said introduced alteration is a knockout of endogenous LGR4, LGR5 or LGR7 gene expression.

18. A method of screening a sample for the presence of a ligand for a receptor selected from the group consisting of LGR4, LGR5 and LGR7, said method comprising:
contacting said sample with a receptor selected from the group consisting of LGR4, LGR5 and LGR7 or a mimetic thereof, and
detecting the presence of a binding event between said receptor and ligand in said sample.
